(19) United States
(12) Patent Application Publication
(10) Pub. No.: US 2024/0074963 A1
(43) Pub. Date: Mar. 7, 2024

(54) SOLID COSMETIC FOR LIPS

(71) Applicant: Shiseido Company, Ltd., Tokyo (JP)

(72) Inventors: Keisuke HAYASHIDA, Tokyo (JP); Kiriko CHIBA, Tokyo (JP); Yusuke NAKANO, Tokyo (JP); Hiroshi OKAMOTO, Tokyo (JP)

(73) Assignee: Shiseido Company, Ltd., Tokyo (JP)

(21) Appl. No.: 18/270,135

(22) PCT Filed: Jan. 26, 2022
(86) PCT No.: PCT/JP2022/002838

§ 371 Date: Jun. 28, 2023

(30) Foreign Application Priority Data
Jan. 28, 2021 (JP) 2021-011975

(51) Int. Cl. A61K 8/85; A61K 8/02; A61K 8/81; A61K 8/895; A61Q 1/06

(52) U.S. Cl. CPC A61K 8/85; A61K 8/0229; A61K 8/8111; A61K 8/895; A61Q 1/06

(57) ABSTRACT

The present invention is to provide a solid lip cosmetic which is excellent in secondary adhesion resistance effect and long lasting performance and has a good feeling of adhesion.

A solid lip cosmetic with secondary adhesion resistance effect and has a good feeling of adhesion can be prepared by blending a copolymer containing a dimer acid ester, and/or a dimer acid ester as an oil component adhering to lips (adhesive oil component), and a phenyl-modified silicone as an oil component seeping into the surface upon application to the lips (seeping oil component).

### RELATED APPLICATION

The present application claims priority to Japanese Patent Application No. 2021-011975 filed Jan. 28, 2021, the disclosure of which is hereby incorporated by reference.

### FIELD OF THE INVENTION

The present invention relates to a solid lip cosmetic, in particular to an improvement of a solid lip cosmetic which is excellent in secondary adhesion resistance effect and long lasting performance and has a good feeling of adhesion.

### BACKGROUND OF THE INVENTION

Conventional lip cosmetics have a problem of secondary adhesion, which is transference of the lipstick to things that come into contact with the lips such as a cup after application of the lipstick to the lips. In order to respond to this problem, lip cosmetics having so-called secondary adhesion resistance effect, which are less likely to cause secondary adhesion, have been developed.

For example, Patent Literatures 1 to 4 disclose a solid lip cosmetic using hydrogenated polyisobutene which adheres to lips and an organic silicone oil less compatible to hydrogenated polyisobutene at room temperature, which cosmetic is homogeneous in the form of a product but separates into hydrogenated polyisobutene and organic silicone oil when shared in use.

With lip cosmetics having the secondary adhesion resistance effect, hydrogenated polyisobutene adheres to the lips in use, and the organosilicon oil layer forms a film over the hydrogenated polyisobutene layer. Since this organosilicon oil is colorless and transparent, even if it is transferred onto a cup or other objects, the stain will not be noticeable, and the secondary adhesion resistance effect will be achieved.

However, when high viscosity oil agents such as hydrogenated polyisobutene and polyisobutylene are blended in large amounts as described above, there are drawbacks of difficulty in spreading during use and a sticky finish.

Then Patent Literature 5 discloses an oil-in-oil lip cosmetic with little stickiness and an excellent secondary adhesion resistance effect, in which a specific amount of phenyl-modified silicone is combined with high viscosity non-volatile ester oil which is not compatible with phenyl-modified silicone even at high temperature, dextrin fatty acid ester and volatile hydrocarbon.

However, even these cosmetics have the problem of stickiness and reduction of the secondary adhesion resistance effect depending on which oil is selected.

### CITATION LIST

### Patent Literature

[Patent Literature 1] Japanese Patent No. 4757950
[Patent Literature 2] Japanese Patent No. 5280490
[Patent Literature 3] Japanese Patent Laid-Open No. 2012-82188
[Patent Literature 4] Japanese Patent No. 6184454
[Patent Literature 5] Japanese Patent No. 6050679

### SUMMARY OF INVENTION

### Problem to be Solved by the Invention

The present invention has been made in view of the above problem in prior art and an object of the present invention is to provide a solid lip cosmetic which is excellent in secondary adhesion resistance effect and long lasting performance and has a good feeling of adhesion.

### Means to Solve the Problem

The present inventors have conducted intensive studies to solve the above problem, and as a result have found that a solid lip cosmetic with a good feeling of adhesion can be prepared by blending a copolymer containing a dimer acid ester, and/or a dimer acid ester as an oil component adhering to lips (adhesive oil component), and a phenyl-modified silicone that separates from the adhesive oil component at room temperature as an oil component seeping into the surface upon application to the lips (seeping oil component).

To achieve the above object, the solid lip cosmetic of the present invention includes the following 5 aspects.
(1) The solid lip cosmetic comprises the following components (A) to (C):
(A) 15 to 50% by mass of (a1) a copolymer containing a dimer acid ester and/or (a2) a dimer acid ester;
(B) 20 to 70% by mass of a phenyl-modified silicone compatible with (A) at 100° C.; and
(C) 5 to 15% by mass of a wax.

It is preferable that the total amount of the component (A), the component (B) and the component (C) accounts for 80 to 100% by mass of the whole cosmetic.

It is preferable that the cosmetic further comprises (D) a color material.

Furthermore, the component (B) separates from the component (A) at 25° C.
(2) In the above cosmetic, the component (B) has a viscosity of 300 mPa·s or less at room temperature.
(3) In the above cosmetic, the ratio between the component (A) and the component (B) is 1:0.5 to 1:4.
(4) In the above cosmetic, the component (A) comprises one or more selected from polyglyceryl-2 isostearate/dimer dilinoleate copolymer and dimer dilinoleyl dimer dilinoleate.
(5) In the above cosmetic, the component (B) comprises one or more selected from diphenylsiloxy phenyl trimethicone and trimethyl pentaphenyl trisiloxane.

### Effect of the Invention

The solid lip cosmetic of the present invention is prepared by blending a specific amount of (A) (a1) a copolymer containing a dimer acid ester and/or (a2) a dimer acid ester, (B) a phenyl-modified silicone that separates from (A) at room temperature and (C) a wax, and therefore has a secondary adhesion resistance effect, achieves a feeling of adhesion and is good in long lasting performance.

### DESCRIPTION OF THE PREFERRED EMBODIMENTS

The solid lip cosmetic of the present invention is composed of (A) (a1) a copolymer containing a dimer acid ester and/or (a2) a dimer acid ester, (B) a phenyl-modified silicone that separates from (A) at room temperature, and (C) a wax.

Hereinafter the respective components will be described in detail.

(A) (a1) Copolymer Containing Dimer Acid Ester/(a2) Dimer Acid Ester

The copolymer containing a dimer acid ester (a1) is a high viscosity non-volatile oil. To improve the secondary adhesion resistance effect, those which are compatible with phenyl-modified silicone (B) at 100° C. and separate therefrom at 25° C. are selected. The dimer acid ester (a2) is liquid fatty acid ester containing, as the main component, a dibasic acid of C36 dicarboxylic acid formed by dimerization of C18 unsaturated fatty acid made of vegetable oils and fats as the raw material, and a monobasic acid and a tribasic acid.

Examples of copolymers containing a dimer acid ester (a1) include a commercially available product, HAILUCENT ISDA (made by Kokyu Alcohol Kogyo Co., Ltd.). Examples of dimer acid esters (a2) include a commercially available product, LUSPLAN DD-DA (made by Nippon Fine Chemical Co., Ltd.).

It is preferable that the amount of the component (A) blended is 15 to 50% by mass based on the whole cosmetic.

The amount of the component (A) blended is preferably 20 to 40% by mass, and more preferably 25 to 35% by mass from the viewpoint of adhesiveness to lips and durability of the cosmetic effect.

(B) Phenyl-Modified Silicone

It is preferable that phenyl-modified silicone (B) has a viscosity of 300 mPa·s or less. More preferably phenyl-modified silicone has a viscosity of 200 mPa·s or less. In the present invention, viscosity is measured by a B-type viscometer TVB-10 (made by Yamato Scientific Co., Ltd.) at 25° C. with spindle No. M1 at 100 rpm.

Phenyl-modified silicone is compatible with the copolymer containing a dimer acid ester (A) (a1) and/or the dimer acid ester (a2) when mixed at 100° C. and separates therefrom at 25° C.

Condition of Separation

In the present invention, whether the components are “separated” or not is observed under the following conditions.

(Measurement Conditions)

The component (A) and the component (B) are heated to 100° C. and mixed with stirring at (A):(B)=1:1 (mass ratio) and allowed to stand. Those which are separated into two homogeneous layers at the boundary when the temperature of the mixture reaches 25° C. are rated as “separated” and those which are transparent and have no boundary are rated as “not separated.” Those which are opaque without visible boundaries are rated as cloudy.

In the present invention, whether components are “compatible” or not is determined based on the following condition.

Components are determined as compatible when the mixture is transparent without visible boundaries at 100° C., in other words, “not separated.”

Examples of phenyl-modified silicone (B) include trimethyl pentaphenyl trisiloxane, diphenyl dimethicone, diphenylsiloxy phenyl trimethicone and phenyl trimethicone.

Trimethyl pentaphenyl trisiloxane and diphenylsiloxy phenyl trimethicone are preferred as the phenyl-modified silicone (B) according to the present invention.

Examples of trimethyl pentaphenyl trisiloxane include commercially available methyl phenyl silicone PH-1555 (made by Dow Toray Co., Ltd.).

Examples of diphenylsiloxy phenyl trimethicone include commercially available Silicone KF56 (made by Shin-Etsu Chemical Co., Ltd.).

The amount of the component (B) blended is 20 to 70% by mass based on the whole cosmetic. The amount of the component (B) blended is preferably 30 to 55% by mass, and more preferably 35 to 50% by mass.

When the amount of the component (B) blended is less than 20% by mass, the cosmetic is less likely to be separated when applied and there is little secondary adhesion resistance effect. Furthermore, when the amount of the component (B) blended is more than 70% by mass, the amount of other components blended is reduced, and there is little secondary adhesion resistance effect.

The component (B) has a viscosity of 300 mPa·s or less.

When the component (B) has a viscosity of more than 300 mPa·s, the component (B) is no longer compatible with the adhesive oil component at high temperature, and thus wax (C) is not homogeneously dissolved, and the blend cannot be molded.

Ratio Between Component (A) and Component (B)

The ratio of blending between the component (A) and the component (B) is preferably 1:0.5 to 1:4. The ratio of blending is more preferably 1:0.8 to 1:3.5. When the ratio of blending is in that range, the component (A) and the component (B) are compatible at 100° C. and separate at 25° C.

(C) Wax

The wax (C) blended to the solid lip cosmetic of the present invention is not particularly limited as long as it is usually blended in cosmetics.

It is preferable that the wax (C) used in the present invention is compatible with the component (A) and the component (B) at high temperature and separate therefrom at room temperature.

Examples of wax (C) used in the present invention include carnauba wax, candelilla wax, polyethylene wax, beeswax, ceresin, microcrystalline wax, solid paraffin, Japan wax, beeswax and polyethylene wax.

The amount of the component (C) blended is preferably 5 to 15% by mass based on the total amount of the cosmetic.

The amount of the component (C) blended is more preferably 7 to 12% by mass. When the amount of the component (C) blended is 5% by mass or less, the resultant may be difficult to be solidified. When the amount of the component (C) blended is more than 15%, the resulting cosmetic may be difficult to be spread and less glossy.

The solid lip cosmetic of the present invention may include a component usually used for solid lip cosmetics as an optional component in addition to the above essential components (A) to (C).

It is also preferable to blend a color material (D) in the solid lip cosmetic of the present invention. A color material usually used for solid lip cosmetics may be blended as the color material.

The color material (D) may be one usually used for cosmetics, and may be in the form of powder or lake (with oil kneaded into it). The color material (D) may be an inorganic pigment, an organic pigment or a pearl pigment.

In this case it is desired that the color material is dissolved or dispersed in the component (A) which adheres to lips and is not dissolved or dispersed in the component (B) which is the seeping oil component. This is because when the color material is also dispersed in the component (B), the secondary adhesion resistance effect is reduced.

A pigment, a pearl pigment, a lame agent and an agent prepared by chelating them, which are usually blended in cosmetics, may be used as the color material.

Examples of color materials include an inorganic white pigment (titanium dioxide, zinc oxide), an inorganic red pigment (iron oxide (red iron oxide), iron titanate), an inorganic brown pigment (γ-iron oxide), an inorganic yellow pigment (yellow iron oxide, yellow ocher), an inorganic black pigment (black iron oxide, carbon, titanium suboxide), an inorganic purple pigment (mango violet, cobalt violet), an inorganic green pigment (chromium oxide, chromium hydroxide, cobalt titanate), an inorganic blue pigment (ultramarine, Prussian blue), a pearl pigment (titanium oxide-coated mica, titanium oxide-coated bismuth oxychloride, titanium oxide-coated talc, colored titanium oxide-coated mica, bismuth oxychloride, fish scale guanine), a metal powder pigment (aluminum powder, copper powder), an organic pigment (Red No. 202, Red No. 205, Red No. 220, Red No. 228, Red No. 405, Orange No. 203, Orange No. 204, Yellow No. 205, Yellow No. 40, Blue No. 404), zirconium, barium, aluminum lake organic pigment (Red No. 3, Red No. 104, Red No. 227, Red No. 401, Orange No. 205, Yellow No. 4, Yellow No. 20, Green No. 3, Blue No. 1), a natural coloring (chlorophyll, carcinoid (β-carotene), carthamin, cochineal, calcone, curcumin, betanine, flavonol, flavone, anthocyanidin, anthraquinone, naphthoquinone) and a functional pigment (boron nitride, photochromic pigment, synthetic fluorphlogopite, iron-containing synthetic fluorphlogopite and hybrid fine powder).

The amount of the component (D) blended is preferably 0.1 to 15% by mass based on the whole cosmetic. The amount of the component (D) blended is more preferably 4 to 10% by mass. When the amount of the component (D) blended is less than 0.1% by mass, the secondary adhesion resistance effect is unlikely to be felt in some cases. When the amount of the component (D) blended is more than 15% by mass, the amount of the components (A) to (C) is relatively reduced, and thus the secondary adhesion resistance effect may be reduced.

The solid lip cosmetic of the present invention may be prepared without so-called “binder oil.” The binder oil allows the adhesive oil component and the steeping oil component to be compatibilized at high temperature. In the present invention, the cosmetic does not necessarily include, but may include, the binder oil.

Examples of binder oils include diisostearyl malate, neopentyl glycol dicaprate, triethylhexanoin, pentaerythrityl tetraethylhexanonate, caprylic/capric triglyceride. It is preferable to blend 0 to 20% by mass of the binder oil.

In addition to the above components, a component used for usual lip cosmetics such as an oil agent, powder, a polymer compound, a moisturizer, a perfume, an antioxidant, a preservative and a beauty ingredient may be blended in the solid lip cosmetic of the present invention to the extent that the effect of the present invention is not lost.

In the present invention it is preferable that the amount of powder added is 30% by mass or less. The amount of powder added is more preferably 20% by mass or less. When the amount of powder added is more than 30% by mass, the amount of the components (A) to (C) blended is relatively reduced, and thus the secondary adhesion resistance effect may be reduced.

Examples of powders include a spherical powder, a flaky powder and a color material.

Examples of spherical powders include a spherical resin powder such as methyl polymethacrylate, organopolysiloxane elastomer, polystyrene, polyamide resin (nylon), polyethylene, a copolymer of styrene and acrylic acid, benzoguanamine resin, polytetrafluoroethylene and a silicone resin.

Examples of flaky powders include inorganic powder such as mica, synthetic mica, talc, sericite, aluminum oxide, magnesium oxide, zirconium oxide, magnesium carbonate, calcium carbonate, calcium sulfate, chromium oxide, chromium hydroxide, aluminum silicate, magnesium silicate, aluminum magnesium silicate, kaolin, silicon carbide, barium sulfate, bentonite, smectite and boron nitride, an organic powder such as N-acyl lysine, and a composite powder such as fine particle titanium oxide-coated titanated mica, fine particle zinc oxide-coated titanated mica, and barium sulfate-coated titanated mica.

A hydrophilized powder may also be used as the powder such as a pigment, a pearl pigment and a lame agent. Powder prepared by a hydrophilization treatment known in the art may be used as the hydrophilized powder. Both organic treatment and inorganic treatment may be used as the hydrophilization treatment. The hydrophilizing agent is not particularly limited and examples thereof include polyhydric alcohol, polysaccharide, water-soluble polymer, metal alkoxide and water glass.

It is preferable that the components of the solid lip cosmetic of the present invention are designed so that the components are in a homogeneous phase without separation throughout the process of production.

The solid lip cosmetic of the present invention may be applied to a lipstick, a lip gloss, a lip primer, an overcoat for lipsticks, and a lip cream. In particular, a lipstick in the form of a solid stick is preferred.

### EXAMPLES

The present invention will be described with reference to Examples below, but the present invention is not limited by those Examples. The amount blended is in % by mass unless otherwise specified.

First, the present inventors observed compatibility of a copolymer containing a dimer acid ester (A) (a1) and/or a dimer acid ester (a2) with phenyl-modified silicone (B). The results are shown in the following Table 1.

(Conditions of Measurement of Mixed State)

The component (A) and the component (B) were heated to 90° C. and mixed with stirring at (A):(B)=1:1 (mass ratio), and then the condition was observed. Next the mixture was allowed to stand, and the condition was also observed when the temperature of the mixture reached 25° C. Those which were separated into two homogeneous layers at the boundary were rated as “separated.” Those which were transparent and had no boundary were rated as “compatible.” Those which were opaque without visible boundaries were rated as “cloudy”.

TABLE 1

| | | Phenyl-modified silicone | | |
|---|---|---|---|---|
| Compatibility of oil component at | | Diphenylsiloxy phenyl | Trimethyl pentaphenyl | Diphenyl |
| 100° C. and 25° C. (100° C./25° C.) | | trimethicone * 1 | trisiloxane * 2 | dimethicone * 3 |
| High | Polyglyceryl-2 isostearate/dimer | Compatible/separated | Compatible/separated | Separated/separated |
| viscosity | dilinoleate copolymer(HAILUCENT | | | |
| ester oil | ISDA)*4 | | | |
| | Dimer dilinoleyl dimer | Compatible/separated | Compatible/separated | Separated/separated |
| | dilinoleate * 5 | | | |
| | 1:1 Mixture of polyglyceryl-2 | Compatible/separated | Compatible/separated | Separated/separated |
| | isostearate/dimer dilinoleate | | | |
| | copolymer and dimer dilinoleyl | | | |
| | dimer dilinoleate | | | |
| | Di(octyldodecyl/phytosteryl/ | Compatible/solidified | Compatible/solidified | Separated/separated |
| | behenyl) lauroyl glutamate * 6 | | | |
| | 1:1 Mixture of polyglyceryl-2 | Compatible/cloudy | Compatible/solidified | Separated/separated |
| | isostearate/dimer dilinoleate | | | |
| | copolymer and | | | |
| | di(octyldodecyl/phytosteryl/ | | | |
| | behenyl) lauroyl glutamate | | | |

* 1 Silicone KF56 (Shin-Etsu Chemical Co., Ltd.)
* 2 Methyl phenyl silicone PH-1555 (Dow Corning Corporation)
* 3 Silicone KF54 (Shin-Etsu Chemical Co., Ltd.)
*4HAILUCENT ISDA (Kokyu Alcohol Kogyo Co., Ltd.)
* 5 LUSPLAN DD-DA7 (Nippon Fine Chemical Co., Ltd.)
* 6 ELDEW PS-306 (Ajinomoto Co., Inc.)

The results of Table 1 shows that only the copolymer containing a dimer acid ester (a1), the dimer acid ester (a2), and the 1:1 mixture of the copolymer containing a dimer acid ester (a1) and the dimer acid ester (a2) were compatible with phenyl-modified silicone at 100° C. and separated therefrom at 25° C. Of the phenyl-modified silicones (B), diphenyl dimethicone was incompatible with the component (A) at 100° C.

The present inventors prepared samples (solid lipsticks) having the composition shown in the tables by a usual method. The respective samples were evaluated according to the following criteria.

(Method of Evaluating Moldability)

Upon removal of the lipstick from the mold for molding, evaluation was made according to the following criteria.
A: Lipstick not chipped, or not adhering to mold
C: Lipstick chipped, or adhering to mold

(Method of Evaluating Secondary Adhesion Resistance)

A practical usability test was performed by a panel of 10 experts. The secondary adhesion resistance effect when the sample was applied to lips was assessed (scored) on a scale of 1 to 5 based on the following grading scale. From the average score, determination was made based on the following evaluation criteria.

(Score)
5: Very good
4: Good
3: Moderate
2: Poor
1: Bad

(Evaluation Criteria)
S: Average score of 4.0 or more
A: Average score of 3.5 or more and less than 4.0
B: Average score of 2.5 or more and less than 3.5
C: Average score of less than 2.5

(Method of Evaluating Gloss)

A practical usability test was performed by a panel of 10 experts. Gloss when the sample was applied to lips was assessed (scored) on a scale of 1 to 3 based on the following grading scale. From the average score, determination was made based on the following evaluation criteria.

(Score)
3: Glossy
2: Moderate
1: Mat

### (Evaluation Criteria)

A: Average score of 2.0 or more
B: Average score of 1.5 or more and less than 2.0
C: Average score of less than 1.5

(Method of Evaluating Long Lasting Performance of Cosmetic)

A practical usability test was performed by a panel of 10 experts. Long lasting performance of the cosmetic two hours after the sample was applied to lips was assessed (scored) on a scale of 1 to 3 based on the following grading scale. From the average score, determination was made based on the following evaluation criteria.

(Score)
3: Long lasting performance superior to the reference formulation.
2: Long lasting performance slightly superior to the reference formulation.
1: Almost the same or lower long lasting performance than the reference formulation.

### (Evaluation Criteria)

A: Average score of 2.0 or more
B: Average score of 1.5 or more and less than 2.0
C: Average score of less than 1.5

[Reference Formulation]

### Lipstick Including Hydrogenated Polyisobutene as Adhesive Oil Component

| Component | % by mass |
|---|---|
| Hydrogenated polyisobutene | 20 |
| Diphenylsiloxy phenyl trimethicone | 9 |
| Trimethyl pentaphenyl trisiloxane | 40 |
| Diphenyl dimethicone | 9 |
| Sorbitan sesquiisostearate | 4 |
| Polyethylene wax | 7 |
| Color material | 11 |

The present inventors investigated phenyl-modified silicones (B) that can be used as the steeping oil component.

The results are shown in Table 2.

TABLE 2

| | Diphenylsiloxy phenyl | Trimethyl pentaphenyl | Diphenyl |
|---|---|---|---|
| Name of phenyl silicone | trimethicone * 1 | trisiloxane * 2 | dimethicone * 3 |
| Viscosity of phenyl-modified silicone | 50 | 175 | 390 |
| (A) Polyglyceryl-2 isostearate/dimer | 30 | 30 | 30 |
| dilinoleate copolymer * 4 | | | |
| (B) Phnyl-modified silicone | 50 | 50 | 50 |
| (C) Wax | 10 | 10 | 10 |
| (D) Color material | 10 | 10 | 10 |
| Moldability | A | A | C |
| Secondary adhesion resistance effect | A | A | — |
| Gloss | A | A | — |
| Long lasting performance | A | A | — |

The results of Table 1 and Table 2 show that when a phenyl-modified silicone (B) which does not satisfy the requirement of “separating from the component (A) at 25° C. and being compatible with the component (A) at 100° C.” is used among the phenyl-modified silicones (B), the resultant does not have good moldability. The results also suggest that phenyl-modified silicone, the component (B), has a viscosity of preferably 300 mPa·s or less. Phenyl-modified silicone has a viscosity of more preferably 200 mPa·s or less.

The present inventors investigated the ratio of mixing between the component (A) and the component (B).

The results are shown in Table 3.

TABLE 3

| Test Example | 1-1 | 1-2 | 1-3 | 1-4 | 1-5 | 1-6 |
|---|---|---|---|---|---|---|
| (A) Polyglyceryl-2 | 60 | 45 | 40 | 30 | 22 | 17.4 |
| isostearate/dimer dilinoleate | | | | | | |
| copolymer*4 | | | | | | |
| (B)Trimethyl pentaphenyl | | | | 55 | | |
| trisiloxane*2 | | | | | | |
| (B)Diphenylsiloxy phenyl | 25 | 30 | 45 | | 63 | 67.6 |
| trimethicone *1 | | | | | | |
| (C) Wax | 10 | 15 | 10 | 10 | 10 | 10 |
| (D) Color material | 5 | 10 | 5 | 5 | 5 | 5 |
| Total | 100 | 100 | 100 | 100 | 100 | 100 |
| (A):(B) | 1:0.4 | 1:0.7 | 1:1.1 | 1:1.8 | 1:2.9 | 1:3.9 |
| Moldability | A | A | A | A | A | A |
| Secondary adhesion resistance | C | A | A | A | A | A |
| effect | | | | | | |
| Gloss | B | A | A | A | A | A |
| Long lasting performance | C | A | A | A | A | A |

The results of Test Examples 1-2 to 1-6 of Table 3 show that when the ratio of blending between the component (A) and the component (B) is 1:0.5 to 1:4, a solid lip cosmetic with a high secondary adhesion resistance effect and excellent usability can be obtained.

The present inventors also studied to find an adhesive oil component other than the copolymer containing a dimer acid ester (A)(a1). The results are shown in Table 4.

TABLE 4

| | | 2-1 | 2-2 | 2-3 | 2-4 | 2-5 | 2-6 | 2-7 |
|---|---|---|---|---|---|---|---|---|
| (A) | Polyglyceryl-2 isostearate/ | 8 | 15 | 20 | — | — | — | 15 |
| | dimer dilinoleate copolymer*4 | | | | | | | |
| | Dimer dilinoleyl dimer | 8 | 15 | 20 | 15 | 30 | — | 15 |
| | dilinoleate*5 | | | | | | | |
| (A′) | Di(octyldodecyl/phytosteryl/ | | | | — | — | 15 | |
| | behenyl) lauroyl glutamate*6 | | | | | | | |
| (B) | (B)Diphenylsiloxy phenyl | 69 | 55 | 45 | 70 | 55 | 70 | — |
| | trimethicone *1 | | | | | | | |
| | Diphenyl dimethicone | | | | | | | 55 |
| (C) | Wax | 10 | 10 | 10 | 10 | 10 | 10 | 10 |
| | Color material | 5 | 5 | 5 | 5 | 5 | 5 | 5 |
| | Total | 100 | 100 | 100 | 100 | 100 | 100 | 100 |
| | Moldability | A | A | A | A | A | A | C |
| | Secondary adhesion resistance | B | A | A | B | A | C | — |
| | effect | | | | | | | |
| | Gloss | A | A | A | A | A | A | — |
| | Long lasting performance | B | A | A | B | A | C | — |

The results of Test Examples 2-1 to 2-3 show that good usability is achieved also when the component (A) includes both polyglyceryl-2 isostearate/dimer dilinoleate copolymer and dimer dilinoleyl dimer dilinoleate. Furthermore, the results of Test Examples 2-4 to 2-5 show that dimer dilinoleyl dimer dilinoleate alone can also work as the adhesive oil component.

### Formulation Example 1: Lip Stick

| | Compounding Amount |
|---|---|
| Component | (% by mass) |
| Polygryceryl-2 isostearated/ | 30 |
| dimer dilinoleate copolymer | |
| Diphenylsiloxy phenyl trimethicone | 44.9 |
| Polyethylene wax | 10 |
| Color material | 10 |
| Pearl pigment | 5 |
| Niacinamide | 0.1 |

### Formulation Example 2: Lip Stick

| | Compounding Amount |
|---|---|
| Component | (% by mass) |
| Polygryceryl-2 isostearated/ | 15 |
| dimer dilinoleate copolymer | |
| Dimer dilinoleyl dimer dilinoleate | 15 |
| Diphenylsiloxy phenyl trimethicone | 45 |
| Polyethylene wax | 10 |
| Color material | 10 |
| Pearl pigment | 5 |

### Formulation Example 3: Lip Stick

| | Compounding Amount |
|---|---|
| Component | (% by mass) |
| Polygryceryl-2 isostearated/ | 30 |
| dimer dilinoleate copolymer | |
| Diphenylsiloxy phenyl trimethicone | Remaining |
| | amount |
| Polyethylene wax | 10 |
| Color material | 10 |
| Pearl pigment | 5 |
| Niacinamide | 5 |

1. A solid lip cosmetic comprising:
(A) 15 to 50% by mass of (a1) a copolymer containing a dimer acid ester and/or (a2) a dimer acid ester, as an adhesive oil;
(B) 20 to 70% by mass of a phenyl-modified silicone that separates from (A) at 25° C. and is compatible with (A) at 100° C., as a seeping oil; and
(C) 5 to 15% by mass of a wax,
wherein a total amount of (A) to (C) accounts for 80 to 100% by mass of the whole cosmetic.

2. The solid lip cosmetic according to claim 1, wherein (B) has a viscosity of 300 mPa·s or less.

3. The solid lip cosmetic according to claim 1, wherein a ratio between (A) and (B) is 1:0.5 to 1:4.

4. The solid lip cosmetic according to claim 1, wherein (A) comprises one or more selected from polyglyceryl-2 isostearate/dimer dilinoleate copolymer and dimer dilinoleyl dimer dilinoleate.

5. The solid lip cosmetic according to claim 1, wherein (B) comprises one or more selected from diphenylsiloxy phenyl trimethicone and trimethyl pentaphenyl trisiloxane.
